(19) United States
(12) Patent Application Publication
(10) Pub. No.: US 2010/0114147 A1
(43) Pub. Date: May 6, 2010

(54) DIRECTIONAL SOFT TISSUE DILATOR AND DOCKING PIN WITH INTEGRATED LIGHT SOURCE FOR OPTIMIZATION OF RETRACTOR PLACEMENT IN MINIMALLY INVASIVE SPINE SURGERY

(75) Inventor: Ashok Biyani, Sylvania, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE UNIVERSITY OF TOLEDO, Toledo, OH (US)

(21) Appl. No.: 12/609,619

(22) Filed: Oct. 30, 2009

Related U.S. Application Data
(60) Provisional application No. 61/109,595, filed on Oct. 30, 2008.

(51) Int. Cl. A61M 29/00; A61B 1/06

(52) U.S. Cl. 606/191; 600/249

(57) ABSTRACT

A soft tissue dilator for use in a surgical procedure includes a first dilator member that defines a first axis and a second dilator member that defines a second axis. The second dilator member is supported on the first dilator member such that relative rotational movement of the first and second dilator members causes eccentric movement of the first and second axes defined by the first and second dilator members. Additional dilators members may be sequentially supported on the first and second dilator members.

### STATEMENTS REGARDING FEDERALLY SPONSORED RESEARCH OR DEVELOPMENT AND CROSS-RELATED APPLICATIONS

This invention was not made with any government support. This application claims the benefit of U.S. Provisional Application No. 61/109,595 filed Oct. 30, 2008, the disclosure of which is expressly incorporated herein by reference.

### BACKGROUND OF THE INVENTION

This invention relates to the field of orthopedic surgery and more particularly to the area of spinal surgery. In particular, this invention relates to improved structures for both a soft tissue dilator and a docking pin for optimizing the placement of a retractor in a minimally invasive spine surgery.

In the past, surgery typically required large incisions to provide visual and instrument access to the surgical site. These large incisions resulted in significant blood loss, damage to muscle tissue, long healing times accompanied by prolonged pain, and significant scarring. Today, however, many surgeries are conducted using minimally invasive techniques. These techniques minimize patient trauma by creating a relatively small incision, followed by the introduction of dilators to increase the effective size of the incision. Following dilation, surgery is performed through a surgical port inserted into the dilated incision. Instead of cutting through the muscle surrounding the surgical site, dilation effectively splits the muscle. Splitting, rather than cutting, the muscle causes less damage to the muscle and leads to faster recovery times and reduced patient discomfort.

Dilators develop a channel from the subcutaneous layer of a patient to the site of operation. Initially, a small incision is made overlying the surgical area of interest. Then, a solid or cannulated pointed rod is inserted into the incision to penetrate the underlying structures and reach the surgical site. It is best if the rod can be positioned against a bony surface, inasmuch as the subsequent application of the dilators will attempt to push this rod forward. Conventional fluoroscopic techniques may be used before and/or after insertion of the initial rod to confirm placement at the desired surgical site. Thereafter, increasingly larger diameter dilators can then be sequentially inserted over each other to gradually enlarge the size of the channel. The increasingly larger diameters of the sequentially inserted dilators help to dilate the path of exposure, while lessening the magnitudes of the forces needed to create such path. The pointed tips of the dilators ease insertion and help to widen the base of the channel when the dilators are orbited around a central axis formed through the center of the dilator along its length at the level of the skin.

Most current dilators allow symmetric circumferential dilation with a constant center point, which is not always desirable. They also make precise placement of the working port relatively time consuming. If a starting guide wire or cannula is slightly off of the desired starting point, symmetric dilatation may cause stretching of the important structures, such as neural elements posteriorly or may bring final docking of the cannula too far anteriorly. Thus, it would be desirable to provide an improved structures for a soft tissue dilator having a variable center point that avoids these potential issues.

### SUMMARY OF THE INVENTION

This invention is a new dilator system for insertion of cannula and working port for minimally invasive spinal surgery. Such an invention allows preferential anterior, posterior, superior or inferior dilatation for docking of the working port in an anatomically more desirable location. The new dilator system permits preferential dilation of one side of the tissue without excessively stretching the vital tissues in undesirable location. The improved dilator of this invention is interchangeable with symmetric dilators if necessary. It may also be made compatible with available neurophysiologic monitoring tools or illumination systems that are known in the art. The dilator tubes may be either incomplete or fully cylindrical in nature and may have asymmetric wall thickness such that they facilitate preferentially dilate tissues. Once the dilatation is completed, a fully circular or oval or any other desirable shaped working port can be placed and the dilating cannulas removed. The dilating cannulas may also be interchangeable with closed symmetric tubular dilators if desired. This invention also describes a combined light source and a docking pin for retention of the retractor blades in the desired location during the surgical procedure. Such a docking pin having an integrated lighting system eliminates the need for separate illumination source and a wire, pin, or shim to maintain the retractor in place. The combined device achieves both objectives and frees up some working space within the retractor. The docking pin with integrated locking system may be disposable and made of variable lengths to fit all sizes and body habitus.

The present invention describes incomplete or nearly complete cannulas of symmetric or variable thickness to allow preferential dilation of tissues towards more anatomically desirable location during minimally invasive exposures for direct lateral spinal fusion. This method avoids anterior migration of the tube or excessive posterior stretching of neural tissues.

Initially, a small diameter complete cannulated or as solid starter tube is placed with radiographic assistance over the desired location. Preferably the starting tube is cannulated, through which a guide wire may be placed to minimize unwarranted migration during the dilatation process. Once the starter tube is confirmed to be in a satisfactory position, several small complete circular dilators or incomplete dilators of symmetric or variable thickness can be placed sequentially for preferential directional dilatation of the tissues. The disclosed invention may be particularly useful during direct lateral exposure of spine or during posterior lumbar minimally invasive surgical techniques.

During direct lateral approaches, typically a transpsaos approach is utilized with symmetric dilatation of the surgical site access pathway. An asymmetric dilator may facilitate placement of initial dilator anterior to the psoas. The thicker wall of the dilating tube may be placed anteriorly if desired and once the tip is at the level of the disc, it may be rotated back and forth to provide posterior mobilization of the psoas muscle. After the last dilator tube is in place, a working port is placed over it and secured in place with means utilized in prior art. If necessary either prior to docking of the working port or during the dilation process, reverse directional tubes may be placed over the previously inserted dilator cannulas to make the dilated hole more symmetric. The dilators may be made compatible with neurophysiologic monitoring. The dilator may be made of any radiolucent or radio-opaque biomaterial.

Alternatively, the dilators may have symmetric wall thicknesses and projections provided on the inner walls thereof which are of variable height such that they facilitate preferential dilatation of the soft tissues. The projections may be linear in nature across entire length of the dilator tube, or they may be located co-linearly only across certain parts or randomly located through out the inner circumference of the tube. The projections may be in cluster of more than one if desired or may be different geometry.

Asymmetric dilatation may also be useful during lumbar minimally invasive surgery. Several surgeons place a pedicle screw prior to performing interbody work during a transforaminal interbody fusion surgical procedure. This sequence of pedicle screw fixation followed by interbody work is particularly desirable in cases of spondylolisthesis. During such procedures, it will be advantageous to use the percutaneous screw insertion tube as a guide for further asymmetric dilation. In this case, asymmetric dilators are placed sequentially over the distal pedicle screw tube (L5 pedicle screw tube for L4-5 transforaminal interbody fusion, for example), such that the center point of the dilator is placed directly over the disc space, providing straight access to the surgical site. Appropriate port may then be placed and secured in location for further surgical work.

If desired, the asymmetric dilators may be placed directly over a tap or a pedicle finder used to prepare the hole for the pedicle screw. Sequential asymmetric dilator placement may also be used for transforaminal interbody fusion, as described above. It also provides access to the corresponding facet joint (L4-5 facet joint with placement of dilator over tap/pedicle finder in L5 pedicle, for example). Such a technique facilitates placement of transfacet screw or performance of facet fusion.

It should be understood that the disclosed invention may be utilized for other orthopedic and non-orthopedic applications.

Various objects and advantages will become apparent to those skilled in the art from the following detailed description of the preferred embodiments, when read in light of the accompanying drawings.

### BRIEF DESCRIPTION OF THE DRAWINGS

FIG. 1 is a perspective view of a first embodiment of a soft tissue dilator in accordance with this invention.

FIG. 2 is a side elevational view of the first embodiment of the soft tissue dilator illustrated in FIG. 1.

FIG. 3 is an end elevational view of the first embodiment of the soft tissue dilator illustrated in FIGS. 1 and 2.

FIG. 4 is a perspective view of a second embodiment of a soft tissue dilator in accordance with this invention.

FIG. 5 is a side elevational view of the second embodiment of the soft tissue dilator illustrated in FIG. 4.

FIG. 6 is an end elevational view of the second embodiment of the soft tissue dilator illustrated in FIGS. 4 and 5.

FIG. 7 is an end elevational view of a dilator member of a third embodiment of a soft tissue dilator in accordance with this invention.

FIG. 8 is an end elevational view of a portion of a dilator member of a fourth embodiment of a soft tissue dilator in accordance with this invention.

FIG. 9 is an end elevational view of a portion of a dilator member of a fifth embodiment of a soft tissue dilator in accordance with this invention.

FIG. 10 is an end elevational view of a portion of a dilator member of a sixth embodiment of a soft tissue dilator in accordance with this invention.

FIG. 11 is an end elevational view of a portion of a dilator member of a seventh embodiment of a soft tissue dilator in accordance with this invention.

FIG. 12 is an end elevational view of a portion of a dilator member of an eighth embodiment of a soft tissue dilator in accordance with this invention.

FIG. 13 is a perspective view of a ninth embodiment of a soft tissue dilator in accordance with this invention.

FIG. 14 is a side elevational view of the ninth embodiment of the soft tissue dilator illustrated in FIG. 13.

FIG. 15 is an end elevational view of the ninth embodiment of the soft tissue dilator illustrated in FIGS. 13 and 14.

FIG. 16 is a side elevational view of an illuminating docking pin in accordance with this invention.

FIG. 17 is a side elevational view of a portion of an alternative embodiment of the illuminating docking pin illustrated in FIG. 16.

### DETAILED DESCRIPTION OF THE PREFERRED EMBODIMENTS

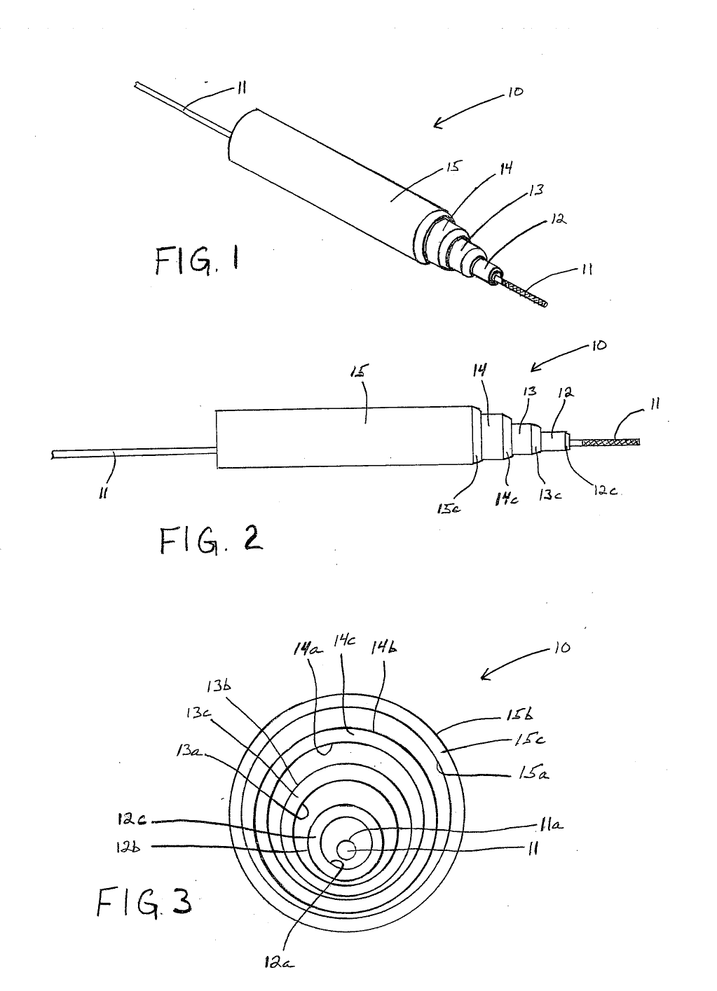

Referring now to the drawings, there is illustrated in FIGS. 1, 2, and 3 a first embodiment of a soft tissue dilator, indicated generally at 10, in accordance with this invention. The first embodiment of the dilator 10 includes a first dilator member 11 that, in the illustrated embodiment, is solid and generally cylindrical in shape and defines an axis. The illustrated first dilator member 11 includes an outer surface 11a that defines a generally circular cross-sectional shape, as best shown in FIG. 3. However, the first dilator member 11 need not be solid (it may be cannulated, for example) and may be formed having any desired cross-sectional shape. The illustrated first dilator member 11 has a leading end surface 11b that is flat and circular in shape. However, the leading end surface 11b may be formed having any desired shape (such as a tapered point) or combination of shapes (such as a combination of flat and tapered surfaces).

The first embodiment of the dilator 10 also includes a second dilator member 12 that is disposed about and supported on the first dilator member 11 for both axial and rotational sliding movement relative thereto. The second dilator member 12 is hollow and includes an inner surface 12a and an outer surface 12b. In the illustrated embodiment, both the inner surface 12a and the outer surface 12b of the second dilator member 12 are each generally cylindrical in shape and, therefore, define respective cross-sectional shapes that are generally circular and define respective axes, as best shown in FIG. 3. However, as also shown in FIG. 3, the inner surface 12a and the outer surface 12b are not oriented concentrically relative to one another. Rather, the wall thickness of the second dilator member 12 varies circumferentially such that the inner surface 12a and the outer surface 12b are oriented eccentrically relative to one another. The purpose for this eccentric orientation will be explained below. In the illustrated embodiment, the inner surface 12a of the second dilator member 12 is disposed about and supported on the outer surface 11a of the first dilator member 11 for both axial and rotational sliding movement relative thereto. The second dilator member 12 is provided with a leading end surface 12c that extends from the outer surface 12b thereof to the inner surface 12a. In the illustrated embodiment, the leading end surface 12c includes an outer tapered portion and an inner flat portion. However, the leading end surface 12b may be formed having any desired shape or combination of shapes.

The first embodiment of the dilator 10 further includes a third dilator member 13 that is disposed about and supported on the second dilator member 12 for both axial and rotational sliding movement relative thereto. The third dilator member 13 is hollow and includes an inner surface 13a and an outer surface 13b. In the illustrated embodiment, both the inner surface 13a and the outer surface 13b of the third dilator member 13 are generally cylindrical in shape and, therefore, define respective cross-sectional shapes that are circular and define respective axes, as best shown in FIG. 3. However, as also shown in FIG. 3, the inner surface 13a and the outer surface 13b are not oriented concentrically relative to one another. Rather, the wall thickness of the third dilator member 13 varies circumferentially such that the inner surface 13a and the outer surface 13b are oriented eccentrically relative to one another. The purpose for this eccentric orientation will be explained below. In the illustrated embodiment, the inner surface 13a of the third dilator member 13 is disposed about and supported on the outer surface 12a of the second dilator member 12 for both axial and rotational sliding movement relative thereto. The third dilator member 13 is provided with a leading end surface 13c that extends from the outer surface 13b thereof to the inner surface 13a. In the illustrated embodiment, the leading end surface 13c includes an outer tapered portion and an inner flat portion. However, the leading end surface 13b may be formed having any desired shape or combination of shapes.

The first embodiment of the dilator 10 additionally includes both a fourth dilator member 14 and a fifth dilator member 15. The fourth dilator member 14 is similar in structure and operation to the second and third dilator members 12 and 13, respectively, and includes an inner surface 14a, an outer surface 14b, and a leading end surface 14c. In the illustrated embodiment, the inner surface 14a of the fourth dilator member 14 is disposed about and supported on the outer surface 13a of the third dilator member 13 for both axial and rotational sliding movement relative thereto. Similarly, the fifth dilator member 15 is similar in structure and operation to the second, third, and fourth dilator members 12, 13, and 14, respectively, and includes an inner surface 15a, an outer surface 15b, and a leading end surface 15c. In the illustrated embodiment, the inner surface 15a of the fifth dilator member 15 is disposed about and supported on the outer surface 14a of the fourth dilator member 14 for both axial and rotational sliding movement relative thereto.

As discussed above, the first embodiment of the dilator 10 can be used to facilitate the performance of a surgery using minimally invasive techniques, wherein a relatively small incision is made in a patient. Specifically, the first embodiment of the dilator 10 can be used to increase the effective size of the relatively small incision to facilitate the desired surgical procedures. To accomplish this, the first dilator member 11 is initially inserted through the relatively small incision in a conventional manner until the leading end surface 11a thereof is positioned at a desired surgical site within the patient. X-rays or other conventional fluoroscopic techniques may be used before and/or after insertion of the first dilator member 11 to confirm placement of the leading end surface 11b at the desired surgical site. Thereafter, the second dilator member 12 is inserted axially over the first dilator member 11 to gradually enlarge the size of the channel extending from the incision to the surgical site. The larger diameter of the second dilator 12 helps to dilate the size of the channel, while lessening the magnitude of the force needed to accomplish this. The tapered leading end surface 12c of the second dilator member 12 eases insertion and helps to widen the base of the channel. Similarly, the third, fourth, and fifth dilator members 13, 14, and 15 can be sequentially inserted axially over the second dilator member 12 to further gradually enlarge the size of the channel. It will be appreciated that any number of such dilator members 12 through 15 may be used as deemed necessary for the particular surgical procedure to be performed.

Unlike known dilator members, however, the eccentric shapes of the dilator members 12 through 15 permit asymmetric dilatation of the channel in a quick and easy manner. Such asymmetric dilatation of the channel can be accomplished simply by rotating one or more of the dilator members 12 through 15 relative to one another. Such rotation can be performed either before or after the dilator members 12 through 15 have been inserted axially thereon. The eccentric structures of the dilator members 12 through 15 permit asymmetric dilatation of the channel in a quick and easy manner.

Although the first embodiment of the dilator 10 is disclosed as having five different dilator members 11 through 15, it will be appreciated that this invention may be practiced with a greater or lesser number of such dilator members. Furthermore, although each of the second through fifth dilator members 12 through 15 of the first embodiment of the dilator 10 is disclosed as having eccentric inner and outer surfaces, it will be appreciated that this invention may be practiced with only some (or only one) of such second through fifth dilator members 12 through 15 having eccentric inner and outer surfaces.

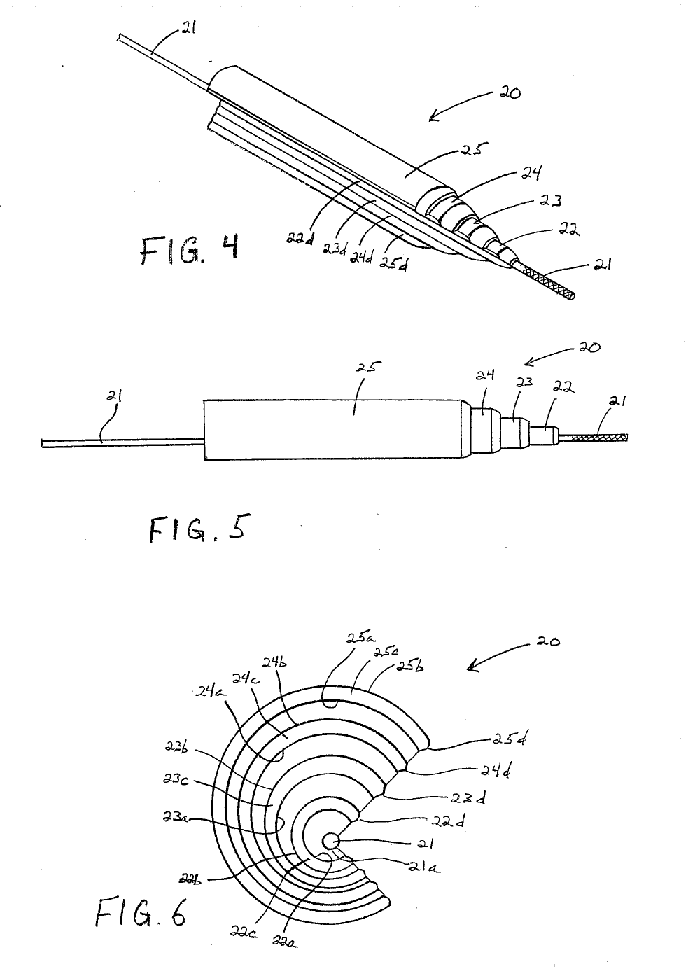

FIGS. 4, 5, and 6 illustrate a second embodiment of a soft tissue dilator, indicated generally at 20, in accordance with this invention. The second embodiment of the dilator 20 is similar to the first embodiment of the dilator 10 and includes a first dilator member 21 that, in the illustrated embodiment, is solid and cylindrical in shape. The illustrated first dilator member 21 includes an outer surface 21 a that defines a circular cross-sectional shape, as best shown in FIG. 6. However, the first dilator member 21 need not be solid and may be formed having any desired cross-sectional shape. The illustrated first dilator member 21 has a leading end surface 21b that is flat and circular in shape. However, the leading end surface 21b may be formed having any desired shape, such as a tapered point.

The second embodiment of the dilator 20 also includes second through fifth dilator members 22 through 25. Similar to the dilator members 12 through 15 of the first embodiment of the dilator 10 described above, the dilator members 22 through 25 of the second embodiment of the dilator 20 have respective inner surfaces 22a through 25a, outer surfaces 22b through 25b, and leading end surfaces 22c through 25c provided thereon. As also described above, some or all of the inner surfaces 22a through 25a of the dilator members 22 through 25 of the second embodiment of the dilator 20 are oriented eccentrically relative to the outer surfaces 22b through 25b thereof. However, unlike the dilator members 12 through 15 of the first embodiment of the dilator 10 described above, the dilator members 22 through 25 of the second embodiment of the dilator 20 are not formed in a circumferentially complete manner. Rather, as best shown in FIG. 6, each of the dilator members 22 through 25 of the second embodiment of the dilator 20 is circumferentially incomplete, having removed portions 22d through 25d provided thereon. The circumferentially incomplete portions 22d through 25d of the dilator members 22 through 25 of the second embodiment of the dilator 20 provide additional space at the surgical site if needed.

FIG. 7 illustrates a dilator member, indicated generally at 30, of a third embodiment of a soft tissue dilator in accordance with this invention. The dilator member 30 is hollow and includes an inner surface 30a and an outer surface 30b. In the illustrated embodiment, both the inner surface 30a and the outer surface 30b of the dilator member 30 are generally cylindrical in shape and, therefore, define respective cross-sectional shapes that are circular. In this embodiment of the invention, however, the inner surface 12a and the outer surface 12b are oriented concentrically relative to one another. Thus, the wall thickness of the dilator member 30 does not vary circumferentially. Rather, a plurality of projections 30c, 30d, 30e, and 30f are provided on the inner surface 30a of the dilator member 30.

In the illustrated embodiment, four of such projections 30c, 30d, 30e, and 30f are provided on the inner surface 30a of the dilator member 30. However, it will be appreciated that this invention may be practiced with any number of such projections, including only a single projection. It will also be appreciated that any number of such projections may be provided on the outer surface 30b of the dilator member 30, either alone or in combination with projections provided on the inner surface 30a of the dilator member 30. Furthermore, the projections 30c, 30d, 30e, and 30f may extend axially throughout some or all of the length of the dilator member 30 as desired.

The projections 30c, 30d, 30e, and 30f are sized to provide an eccentric orientation relative to another dilator member (not shown) when the dilator member 30 is inserted axially over such other dilator member in the manner described above. In the illustrated embodiment, the projection 30c has a first size, the projections 30d and 30e each have a second size that is smaller than the first size, and the projection 30f has a third size that is smaller than the second size. Thus, when the dilator member 30 is inserted axially over another dilator member in the manner described above, the projections 30c, 30d, 30e, and 30f support the dilator member 30 eccentrically relative to such other dilator member. The projections 30c, 30d, 30e, and 30f may be sized as desired to accomplish any desired eccentric orientation.

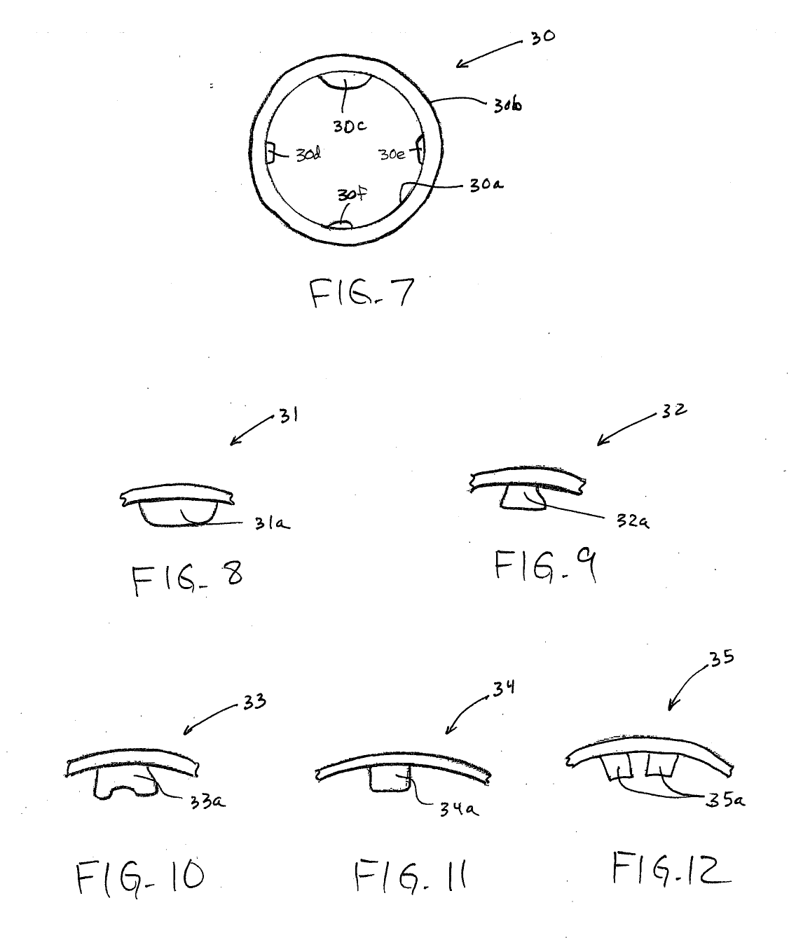

Each of the illustrated projections 30c, 30d, 30e, and 30f has a cross-sectional shape that is generally arcuate. However, it will be appreciated that some or all of the projections 30c, 30d, 30e, and 30f may be different shapes. FIGS. 8 through 12 illustrate some of such alternative shapes. In FIG. 8, a dilator member, indicated generally at 31, includes a projection 31a having a cross-sectional shape that is generally rectangular with convex sides. In FIG. 9, a dilator member, indicated generally at 32, includes a projection 32a having a cross-sectional shape that is generally rectangular with concave sides. In FIG. 10, a dilator member, indicated generally at 33, includes a projection 33a having a cross-sectional shape that is generally rectangular with a central recess. In FIG. 1, a dilator member, indicated generally at 34, includes a projection 34a having a cross-sectional shape that is generally rectangular with radial sides. In FIG. 12, a dilator member, indicated generally at 35, includes a pair of projections 35a, each having a cross-sectional shape that is generally rectangular with radial sides. Any other desired shape or combination of shapes is contemplated to be within the scope of this invention.

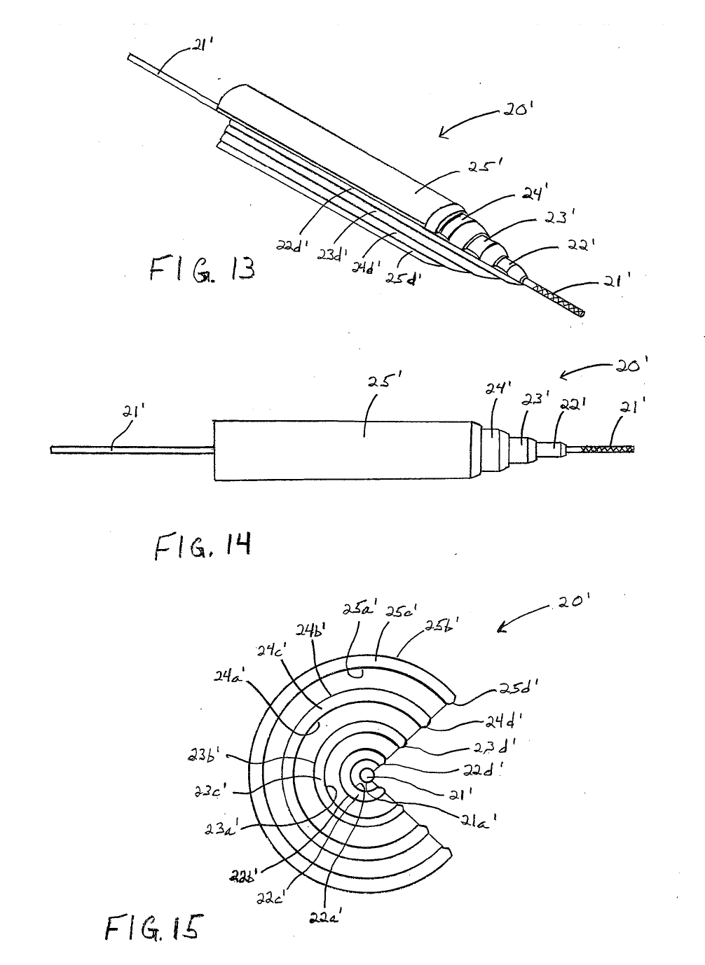

FIGS. 13, 14, and 15 illustrate a ninth embodiment of a soft tissue dilator, indicated generally at 20′, in accordance with this invention. The ninth embodiment of the dilator 20′ is similar to the second embodiment of the dilator 20 and includes a first dilator member 21′ that, in the illustrated embodiment, is solid and cylindrical in shape. The illustrated first dilator member 21′ includes an outer surface 21a′ that defines a circular cross-sectional shape, as best shown in FIG. 15. However, the first dilator member 21′ need not be solid and may be formed having any desired cross-sectional shape. The illustrated first dilator member 21′ has a leading end surface 21b′ that is flat and circular in shape. However, the leading end surface 21b′ may be formed having any desired shape, such as a tapered point.

The ninth embodiment of the dilator 20′ also includes second through fifth dilator members 22′ through 25′. Similar to the dilator members 22 through 25 of the second embodiment of the dilator 20 described above, the dilator members 22′ through 25′ of the ninth embodiment of the dilator 20′ have respective inner surfaces 22a′ through 25a′, outer surfaces 22b′ through 25b′, and leading end surfaces 22c′ through 25c′ provided thereon. However, all of the inner surfaces 22a′ through 25a′ of the dilator members 22′ through 25′ of the ninth embodiment of the dilator 20′ are oriented concentrically relative to the outer surfaces 22b′ through 25b′ thereof. Also, the dilator members 22′ through 25′ of the ninth embodiment of the dilator 20′ are not formed in a circumferentially complete manner. Rather, as best shown in FIG. 15, each of the dilator members 22′ through 25′ of the ninth embodiment of the dilator 20′ is circumferentially incomplete, having removed portions 22d′ through 25d′ provided thereon. The circumferentially incomplete portions 22d′ through 25d′ of the dilator members 22′ through 25′ of the ninth embodiment of the dilator 20′ provide additional space at the surgical site if needed.

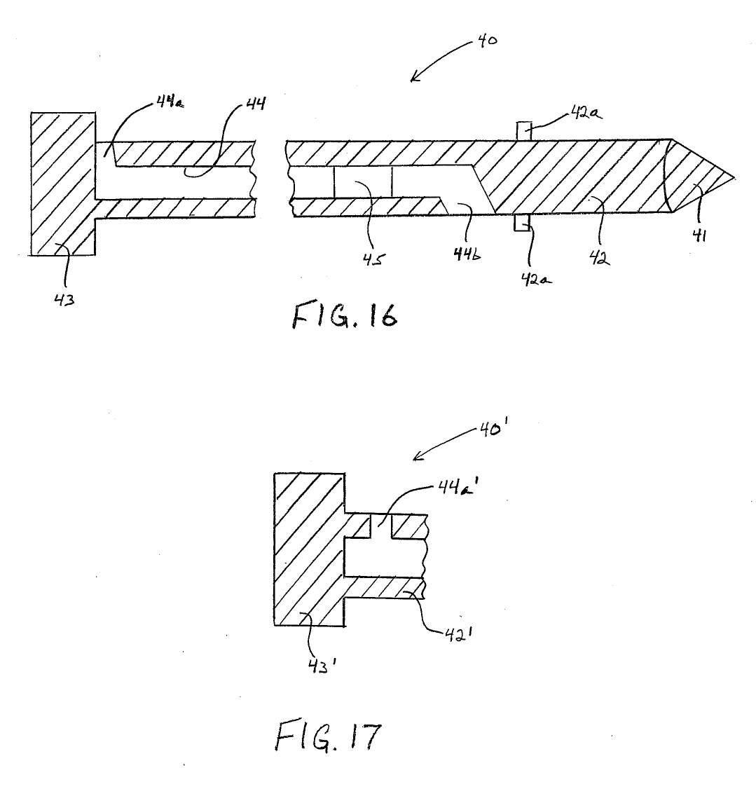

Referring now to FIG. 16, there is illustrated an illuminating docking pin, indicated generally at 40, in accordance with this invention. The illuminating docking pin 40 includes a tip portion 41, an elongated body portion 42, and an end portion 43. The tip portion 41 of the illuminating docking pin 40 is conventional in the art and may be formed having any desired geometry, such as beveled, diamond tip, spear shaped, drill tip, or threaded. The length of the body portion 42 of the illuminating docking pin 40 maybe varied as desired to accommodate the thickness of the tissue between the skin and the lateral aspect of the spinal column. The body portion 42 of the illuminating docking pin 40 may also formed having any desired cross-sectional shape, such as round, oval, trapezoid, etc., and combinations thereof. The end portion 43 of the illuminating docking pin 40 may be utilized for driving the pin 40 in the desired location to secure a retractor (not shown) in place. If desired, the body portion 42 of the illuminated docking pin 40 may have one or more outwardly extending projections 42a provided thereon to prevent the pin 40 from being driven too deep and to help keep the retractor in place.

The illuminating docking pin 40 includes a light source for providing light to the surgical site, which is located near the tip portion 41. The light source of the illuminating docking pin 40 may be embodied as a passageway 44 that extends from a first port 44a located near the end portion 43 to a second port 44b located near the tip portion 41. The second port 44b may, for example, be located from about 1.5 cm to about 5.0 cm away from the tip portion 41 of the illuminating docking pin 40 or elsewhere as desired. The passageway 44 is effective to transmit ambient light from the first port 44a through the body portion 42 and the second port 44b to the surgical site located near the tip portion 41. As shown in FIG. 16, the first port 44a may be located directly adjacent to the end portion 43 of the illuminating docking pin 40. Alternatively, as shown in FIG. 17, the first port 44a′ may be spaced apart from the end portion 43′ within the body portion 42′ of the illuminating docking pin 40′. On the other hand, as also illustrated in FIG. 16, a separate source of light 45 (such as a battery powered lamp) may be disposed within the passageway 44 for this purpose.

The above detailed description of this invention is given for explanatory purposes. It will be apparent to those skilled in the art that numerous changes and modifications other than those cited can be made without departing from the scope of the invention. Accordingly, the whole of the foregoing description is to be construed in an illustrative and not a limitative sense, the scope of the invention being defined by the appended claims.

What is claimed is:

1. A soft tissue dilator for use in a surgical procedure comprising:
a first dilator member that defines a first axis;
a second dilator member that defines a second axis, wherein the second dilator member is supported on the first dilator member such that relative rotational movement of the first and second dilator members causes eccentric movement of the first and second axes defined by the first and second dilator members.

2. The soft tissue dilator defined in claim 1 wherein the first dilator member is generally cylindrical in shape, having an outer surface that defines a generally circular cross-sectional shape.

3. The soft tissue dilator defined in claim 2 wherein the second dilator member is hollow and includes an inner surface and an outer surface, wherein the inner surface of the second dilator member is disposed about and supported on the outer surface of the first dilator member for rotational sliding movement relative thereto.

4. The soft tissue dilator defined in claim 3 wherein the inner surface and the outer surface of the second dilator member are each generally cylindrical in shape and define respective cross-sectional shapes that are generally circular.

5. The soft tissue dilator defined in claim 4 wherein the inner surface and the outer surface of the second dilator member are oriented eccentrically relative to one another.

6. The soft tissue dilator defined in claim 3 wherein the inner surface and the outer surface of the second dilator member are oriented concentrically relative to one another, and wherein a protrusion is provided on the inner surface of the second dilator member that is supported on the outer surface of the first dilator member.

7. The soft tissue dilator defined in claim 3 wherein the inner surface and the outer surface of the second dilator member are oriented concentrically relative to one another, and wherein a plurality of protrusions is provided on the inner surface of the second dilator member that is supported on the outer surface of the first dilator member.

8. The soft tissue dilator defined in claim 1 wherein the second dilator member is circumferential complete.

9. The soft tissue dilator defined in claim 1 wherein the second dilator member is circumferential incomplete.

10. The soft tissue dilator defined in claim 2 wherein the second dilator member is disposed about and supported on the first dilator member for both axial and rotational sliding movement relative thereto.

11. The soft tissue dilator defined in claim 1 further including a third dilator member that defines a third axis, wherein the third dilator member is supported on the second dilator member such that relative rotational movement of the second and third dilator members causes eccentric movement of the second and third axes defined by the second and third dilator members.

12. The soft tissue dilator defined in claim 11 wherein the third dilator member is hollow and includes an inner surface and an outer surface, wherein the inner surface of the third dilator member is disposed about and supported on the outer surface of the second dilator member for rotational sliding movement relative thereto.

13. The soft tissue dilator defined in claim 12 wherein the inner surface and the outer surface of the third dilator member are oriented eccentrically relative to one another.

14. The soft tissue dilator defined in claim 12 wherein the inner surface and the outer surface of the third dilator member are oriented concentrically relative to one another, and wherein a protrusion is provided on the inner surface of the third dilator member that is supported on the outer surface of the second dilator member.

15. The soft tissue dilator defined in claim 11 further including a fourth dilator member that defines a fourth axis, wherein the fourth dilator member is supported on the third dilator member such that relative rotational movement of the third and fourth dilator members causes eccentric movement of the third and fourth axes defined by the third and fourth dilator members.

16. The soft tissue dilator defined in claim 15 wherein the inner surface and the outer surface of the fourth dilator member are oriented eccentrically relative to one another.

17. The soft tissue dilator defined in claim 15 wherein the inner surface and the outer surface of the fourth dilator member are oriented concentrically relative to one another, and wherein a protrusion is provided on the inner surface of the fourth dilator member that is supported on the outer surface of the third dilator member.

18. An illuminating docking pin for use in a surgical procedure comprising:
a tip portion;
an elongated body portion;
an end portion; and
a light source provided in the elongated body portion for providing light near the tip portion.

19. The illuminating docking pin defined in claim 18 wherein the light source is a passageway that extends through the elongated body portion from a first port that is located near the end portion to a second port that is located near the tip portion.

20. The illuminating docking pin defined in claim 18 wherein the light source is a source of light that is disposed in a passageway that extends through the elongated body portion to a port that is located near the tip portion.
